(19) United States
(12) Patent Application Publication
(10) Pub. No.: US 2008/0171894 A1
(43) Pub. Date: Jul. 17, 2008

(54) Mmdi and Pmdi Production By Means of Gas Phase Phosgenation

(75) Inventors: Christian Muller, Mannheim (DE); Eckhard Stroefer, Mannheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/908,422

(22) PCT Filed: Mar. 22, 2006
(86) PCT No.: PCT/EP06/60939

§ 371 Date: Sep. 12, 2007

(30) Foreign Application Priority Data
Mar. 30, 2005 (DE) 10 2005 014 847.6

(51) Int. Cl. C07C 263/00

(52) U.S. Cl. 560/347

(57) ABSTRACT

The invention relates to a process for preparing isocyanates, which comprises the steps
(1) preparation of a crude MDA mixture by reaction of aniline with formaldehyde, with the reaction conditions being selected so that the resulting crude MDA can be converted completely into the gas phase,
(2) conversion of the crude MDA mixture from step (1) into the gas phase and
(3) phosgenation of crude MDA in the gas phase to give MMDI and PMDI.

The invention relates to a process for preparing isocyanates, which comprises the steps
(1) preparation of a crude MDA mixture comprising MMDA and PMDA by reaction of aniline with formaldehyde, with the reaction conditions being selected so that the resulting crude MDA can be converted into the gas phase,
(2) conversion of the crude MDA mixture from step (1) into the gas phase and
(3) phosgenation of crude MDA in the gas phase to give MMDI and PMDI.

Aromatic isocyanates are important and versatile raw materials for polyurethane chemistry. MDI in particular is one of the most important industrial isocyanates. In the technical field and for the purposes of the present patent application, the general term “MDI” is used as generic term for methylenedi(phenyl isocyanates) and polymethylene-polyphenylene polyisocyanates. The term methylenedi(phenyl isocyanate) comprises the isomers 2,2′-methylenedi(phenyl isocyanate) (2,2′-MDI), 2,4′-methylenedi(phenyl isocyanate) (2,4′-MDI) and 4,4′-methylenedi(phenyl isocyanate) (4,4′-MDI). These isomers are, in the specialist field and for the purposes of the present invention, referred to collectively as “monomeric MDI” or “MMDI”. The term polymethylene-polyphenylene polyisocyanates comprises, in the technical field and for the purposes of the present invention, “polymeric MDI” or “PMDI” comprising higher homologues of monomeric MDI and optionally further comprises monomeric MDI.

In customary industrially relevant production processes, MDI is produced by phosgenation of methylenedi(phenylamine) (MDA). The synthesis occurs in a two-stage process. Firstly, aniline is condensed with formaldehyde to form a mixture of monomeric methylenedi(phenylamines), in the specialist field and for the purposes of the present invention referred to as “MMDA”, and polymethylene-polyphenylene polyamines, in the specialist field and for the purposes of the present invention referred to as “PMDA”, known as crude MDA. The crude MDA usually produced by means of processes of the prior art comprises about 70% of MMDA and is preferably produced at an amine to formaldehyde ratio of about 2.0-2.5.

This crude MDA is subsequently reacted with phosgene in a manner known per se in a second step to give a mixture of the corresponding oligomeric and isomeric methylenedi(phenyl isocyanates) and polymethylene-polyphenylene polyisocyanates, known as crude MDI. Here, the isomer and oligomer composition generally remains unchanged. Part of the 2-ring compounds is then usually separated off in a further process step (e.g. by distillation or crystallization), leaving polymeric MDI (PMDI) having a reduced MMDI content as residue.

The phosgenation of the crude MDA mixture is known to those skilled in the art and is described, for example, in “Chemistry and Technology of Isocyanates” by H. Ulrich, John Wiley Veriag, 1996, and in the references cited therein. However, the processes for preparing crude MDI known from the prior art have numerous disadvantages. Firstly, the space-time yield is undesirably low, for example because of intermediates which precipitate in solid form and react slowly during the preparation, and, secondly, the phosgene holdup in the production plants is undesirably high and the energy requirement for the process is also undesirably high.

It was an object of the invention to provide a process for preparing isocyanates which gives a better space-time yield than the processes known from the prior art. Furthermore, a process which makes a lower phosgene holdup in the production plant possible should be provided. In addition, a process which allows a smaller reactor volume in the phosgenation should be provided. Finally, a process which is advantageous from an energy point of view should be provided.

In particular, it was an object of the invention to provide a process having the above advantages for the preparation of MMDI and PMDI. The product mix of MMDI and PMDI in this process should preferably be shifted more strongly in the direction of MMDI, since MMDI is desired by the market. For the present purposes, the term product mix refers to the composition and amount of PMDI and MMDI produced.

The object has unexpectedly been able to be achieved by the methylenedianiline (MDA) process being modified so that a mixture of MMDA and PMDA which can be converted essentially completely into the gas phase is obtained and is subsequently phosgenated in the gas phase.

The invention accordingly provides a process for preparing isocyanates, in particular MMDI and PMDI, which comprises the steps
(1) preparation of a crude MDA mixture comprising MMDA and PMDA by reaction of aniline with formaldehyde, with the reaction conditions being selected so that the resulting crude MDA can be converted into the gas phase,
(2) conversion of the crude MDA mixture from step (1) into the gas phase and
(3) phosgenation of crude MDA in the gas phase to give MMDI and PMDI.

To carry out the reaction of aniline with formaldehyde described in step (1) to form monomeric methylenedi(phenylamines) (for the purposes of the present invention referred to as “MMDA”) and polymethylene-polyphenylene polyamines (for the purposes of the present invention referred to as “PMDA”), with this mixture of methylenedi(phenylamines) and polymethylene-polyphenylene polyamines being referred to as “crude MDA”, the starting materials are usually mixed in a mixing apparatus. Suitable mixing apparatuses are, for example, mixing pumps, nozzles or static mixers. The starting materials are then reacted in a suitable reaction apparatus, for example in tube reactors, stirred reactors and reaction columns or combinations thereof. The reaction temperature is generally in the range from 20 to 200° C., preferably from 30 to 140° C.

The reaction of step (1) is carried out in the presence of an acid as catalyst, with the catalyst preferably being added in admixture with aniline. Preferred catalysts are mineral acids such as hydrochloric acid, sulfuric acid and phosphoric acid. It is likewise possible to use mixtures of acids. Hydrochloric acid is particularly preferred. If hydrogen chloride is used as catalyst, this can also be used in gaseous form. The amount of catalyst is preferably selected so that a molar ratio of acid/aniline (A/A) of from 0.05 to 0.5, particularly preferably from 0.08 to 0.3, is obtained.

In a preferred embodiment, the reaction of step (1) is carried out in aqueous medium using HCl as catalyst. The reaction can also be carried out in the presence of a solvent. Particularly suitable solvents are ethers, water and mixtures thereof. Examples are dimethylformamide (DMF), tetrahydrofuran (THF) and diethyl isophthalate (DEIP).

Formaldehyde can be supplied to the process of the invention in the form of monomeric formaldehyde and/or in the form of higher homologues, known as poly(oxymethylene) glycols.

The composition of the polyamine mixture produced (crude MDA) is decisively influenced not only by the acid concentration and the temperature but also by the molar ratio of aniline molecules introduced to formaldehyde molecules introduced (A/F ratio) both in the continuous MDA process and the discontinuous MDA process. The greater the A/F ratio selected, the greater the MMDA content of the resulting crude MDA solution. It should be noted in this context that a larger A/F ratio not only leads to a larger proportion of 2-ring molecules (MMDA) but also results in the entire oligomer spectrum of polyamines being shifted in the direction of smaller molecules. For example, the 4-ring MDA content drops by about 80% when the A/F ratio is increased from 2.4 to 5.9.

For the purposes of the present invention, the reaction conditions in step (1) are selected so that the resulting crude MDA can be converted into the gas phase, i.e. the reaction conditions are selected so that the resulting crude MDA has such proportions of MMDA and PMDA that it can be converted into the gas phase, preferably completely into the gas phase. In particular, the aniline to formaldehyde ratio in step (1) is selected so that the resulting crude MDA can be converted into the gas phase.

For the present purposes, “able to be converted into the gas phase” means that the resulting crude MDA can be transformed from the liquid state into the gaseous state under the action of reaction conditions suitable for the phosgenation, in particular pressure and temperature and, if appropriate, ratio of amine mixture to inert medium or phosgene described below under the process step (3).

In the case of, for example, an amine to formaldehyde ratio which is too low, an excessively high proportion of PMDA would be obtained in the crude MDA and the resulting crude MDA would not be able to be converted into the gas phase.

Preference is given to the crude MDA formed in step (1) being able to be converted completely into the gas phase. For the present purposes, “completely” means that not more than 2% by weight, preferably not more than 1% by weight, in particular not more than 0.1% by weight, of a residue which cannot be converted into the gas phase remains.

For the purposes of the present invention, the molar ratio of aniline to formaldehyde in process step (1) is generally 3-10:1, preferably 4-8:1, more preferably 5-7.5:1, in particular 5.5-7:1.

In a preferred embodiment, the process conditions in step (1) of the process of the invention are selected so that the crude MDA mixture formed in step (1) has a proportion of

from 88 to 99.9 percent by weight of MMDA and
from 0.1 to 12 percent by weight of PMDA,
based on the total weight of MMDA and PMDA.

The crude MDA mixture formed in step (1) particularly preferably has a proportion of

from 90 to 99.5 percent by weight of MMDA, in particular from 95 to 99 percent by weight of MMDA, and
from 0.5 to 10 percent by weight of PMDA, in particular from 1 to 5 percent by weight of PMDA,
based on the total weight of MMDA and PMDA.

Furthermore, in a preferred embodiment, the process conditions in step (1) of the process of the invention are selected so that the crude MDA mixture formed in step (1) has a mean functionality of from 2.01 to 2.4, preferably from 2.02 to 2.3, in particular from 2.03 to 2.2. For the present purposes, the mean functionality is the average number of amine groups per amine molecule.

The reaction of aniline with formaldehyde can be carried out either continuously or discontinuously, in a batch or semibatch process.

The crude MDA obtained is converted into the gas phase in step (2) of the process of the invention and phosgenated, i.e. reacted with phosgene, in step (3) of the process of the invention.

For the present purposes, “conversion into the gas phase” (2) means that the amine starting material stream comprising MMDA and PMDA is transformed into the gaseous state under conditions which are described below under step 3. Steps (2) and (3) can be carried out successively or simultaneously, i.e. the amine stream becomes gaseous only as a result of injection into the reactor.

The following applies to the gas-phase phosgenation (3):

The preparation of MMDI and PMDI is usually carried out by reaction of the corresponding primary amines from step (2) (i.e. of MMDA and PMDA) with phosgene, preferably an excess of phosgene. According to the present invention, this process takes place in the gas phase. For the purposes of the present invention, “reaction in the gas phase” means that the starting material streams (i.e. the amine stream and the phosgene stream) react with one another in the gaseous state.

The reaction of phosgene with the amine mixture occurs in a reaction space which is generally located in a reactor, i.e. the reaction space is the space in which the reaction of the starting materials occurs, while the reactor is the technical apparatus which comprises the reaction space. Here, the reaction space can be any customary reaction space which is known from the prior art and is suitable for noncatalytic, single-phase gas reactions, preferably for continuous noncatalytic, single-phase gas reactions, and will withstand the moderate pressures required. Suitable materials for contact with the reaction mixture are, for example, metals such as steel, tantalum, silver or copper, glass, ceramic, enamels or homogeneous or heterogeneous mixtures thereof. Preference is given to using steel reactors. The walls of the reactor can be smooth or profiled. Suitable profiles are, for example, grooves or corrugations.

It is generally possible to use the reactor types known from the prior art. Preference is given to tube reactors.

In the process of the invention, the mixing of the reactants occurs in a mixing apparatus in which the reaction stream passed through the mixing apparatus is subjected to high shear. Preference is given to using a static mixing apparatus or a mixing nozzle located upstream of the reactor as mixing apparatus. Particular preference is given to using a mixing nozzle.

The reaction of phosgene with the amine mixture in the reaction space usually occurs at absolute pressures of from >1 bar to <50 bar, preferably from >2 bar to <20 bar, more preferably from 3 bar to 15 bar, particularly preferably from 3.5 bar to 12 bar, in particular from 4 to 10 bar.

In general, the pressure in the feed lines to the mixing apparatus is higher than the pressure in the reactor indicated above. Depending on the choice of mixing apparatus, this pressure drops. The pressure in the feed lines is preferably from 20 to 1000 mbar, particularly preferably from 30 to 200 mbar, higher than in the reaction space.

The pressure in the work-up apparatus is generally lower than in the reaction space. The pressure is preferably from 50 to 500 mbar, particularly preferably from 80 to 150 mbar, lower than in the reaction space.

Step (3) of the process of the invention can, if appropriate, be carried out in the presence of an additional inert medium. The inert medium is a medium which is present in gaseous form in the reaction space at the reaction temperature and does not react with the starting materials at this temperature. The inert medium is generally mixed with amine and/or phosgene prior to the reaction. For example, it is possible to use nitrogen, noble gases such as helium or argon or aromatics such as chlorobenzene, dichlorobenzene or xylene. Preference is given to using nitrogen as inert medium. Particular preference is given to monochlorobenzene or a mixture of monochlorobenzene and nitrogen.

The inert medium is generally used in such an amount that the molar ratio of inert medium to amine is from >2 to 30, preferably from 2.5 to 15. The inert medium is preferably introduced into the reaction space together with the amine.

In the process of the invention, the temperature in the reaction space is selected so that it is below the boiling point of the highest-boiling amine used, based on the pressure prevailing in the reaction space. Depending on the amine (mixture) used and the pressure set, an advantageous temperature in the reaction space is usually from >200° C. to <600° C., preferably from 280° C. to 400° C.

To carry out step (3), it can be advantageous to preheat the streams of reactants prior to mixing, usually to temperatures of from 100 to 600° C., preferably from 200 to 400° C.

The mean contact time of the reaction mixture in step (3) of the process of the invention is generally from 0.1 second to <5 seconds, preferably from >0.5 second to <3 seconds, particularly preferably from >0.6 second to <1.5 seconds. For the purposes of the present invention, the mean contact time is the period of time from the commencement of mixing the starting materials until they leave the reaction space.

In a preferred embodiment, the dimensions of the reaction space and the flow velocities are selected so that turbulent flow, i.e. flow at a Reynolds number of at least 2300, preferably at least 2700, occurs, with the Reynolds number being calculated using the hydraulic diameter of the reaction space. The gaseous reactants preferably pass through the reaction space at a flow velocity of from 3 to 180 meters/second, preferably from 10 to 100 meters/second.

In the process of the invention, the molar ratio of phosgene to amino groups in the feed is usually from 1:1 to 15:1, preferably from 1.2:1 to 10:1, particularly preferably from 1.5:1 to 6:1.

In a preferred embodiment, the reaction conditions are selected so that the reaction gas at the outlet from the reaction space has a phosgene concentration of more than 25 mol/m³, preferably from 30 to 50 mol/m³. Furthermore, the inert medium concentration at the outlet from the reaction space is generally more than 25 mol/m³, preferably from 30 to 100 mol/m³.

In a particularly preferred embodiment, the reaction conditions are selected so that the reaction gas at the outlet from the reaction space has a phosgene concentration of more than 25 mol/m³, in particular from 30 to 50 mol/m³, and at the same time has an inert medium concentration of more than 25 mol/m³, in particular from 30 to 100 mol/m³.

The reaction volume is usually heated via its exterior surface. To build production plants having a high plant capacity, a plurality of reactor tubes can be connected in parallel.

The process of the invention is preferably carried out in a single stage. For the purposes of the invention, this means that the mixing and reaction of the starting materials occurs in one step and in one temperature range, preferably in the abovementioned temperature range. Furthermore, the process of the invention is preferably carried out continuously.

After the reaction, the gaseous reaction mixture is generally scrubbed with a solvent, preferably at temperatures above 150° C. Preferred solvents are hydrocarbons which are optionally substituted with halogen atoms, for example chlorobenzene, dichloro-benzene, and toluene. Particular preference is given to using monochlorobenzene as solvent. In the scrub, the isocyanate is selectively transferred into the scrub solution. The remaining gas and the scrub solution obtained are subsequently separated into isocyanate(s), solvent, phosgene and hydrogen chloride, preferably by means of rectification. Small amounts of by-products remaining in the isocyanate (mixture) can be separated from the desired isocyanate (mixture) by means of additional rectification or else crystallization.

It is in principle possible to separate the products PMDI and MMDI obtained either completely or partly after the phosgenation. This can occur after or before the work-up. Preference is given to working up the product streams of MMDI and PMDI jointly.

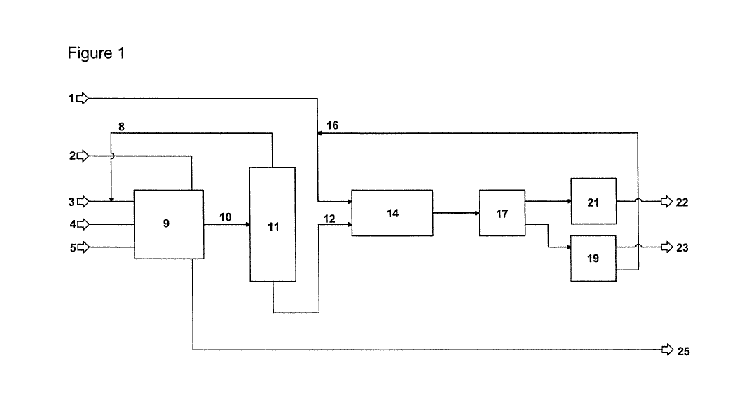

A preferred embodiment of the process of the invention is depicted in FIG. 1.

In FIG. 1:
1 phosgene
2 base
3 aniline
4 formaldehyde
5 hydrochloric acid
8 recirculated aniline
9 MDA reaction space
10 aniline, MMDA, PMDA
11 amine separation
12 MMDA/PMDA mixture
14 reaction space for gas-phase phosgenation
16 recirculated phosgene
17 separation of MMDI/inert medium (e.g. chlorobenzene) from HCl/phosgene/inert medium (e.g. nitrogen)
19 separation of inert medium (e.g. nitrogen) from HCl from phosgene
21 separation of MMDI from inert medium (e.g. chlorobenzene)
22 MMDI/PMDI
23 HCl
25 aqueous salt solution (e.g. NaCl when using HCl and NaOH as base)

The invention further comprises a specific mixture of MMDA and PMDA which is suitable for carrying out the process of the invention. The invention thus provides a mixture comprising monomeric methylenedi(phenylamines) (=MMDA) and polymethylene-polyphenylene polyamines (=PMDA) in which the content of polymethylene-polyphenylene polyamines is so low that the mixture can be converted into the gas phase at temperatures of from 200 to 600° C., preferably at temperatures of from 220° C. to 450° C., and at pressures of from 2 bar to 20 bar, preferably at pressures of from 4 bar to 10 bar.

In a preferred embodiment, the mixture of the invention has a content of from 88 to 99.9 percent by weight of monomeric methylenedi(phenylamines) and from 0.1 to 12 percent by weight of polymethylene-polyphenylene polyamines.

The mixture of the invention particularly preferably has a content of

from 90 to 99.5% by weight of MMDA, in particular from 95 to 99 percent by weight of monomeric methylenedi(phenylamines) and
from 0.5 to 10% by weight of PMDA, in particular from 1 to 5 percent by weight of polymethylene-polyphenylene polyamines.

The invention further provides a gaseous mixture comprising

(a) an amine mixture according to the invention comprising MMDA and PMDA, and
(b) an inert medium.

Suitable inert media are the above-described inert media.

In a preferred embodiment, the components (a) and (b) in the gaseous mixture are used in such amounts that the molar ratio of inert medium to amine is from >2 to 30, preferably from 2.5 to 15.

Finally, the invention provides for the use of a mixture according to the invention according to any of claims 5 to 7 for preparing isocyanates by means of gas-phase phosgenation. The preferred embodiments described for the process of the invention are likewise employed for the use according to the invention.

1. A process for preparing isocyanates, which comprises the steps
(1) preparation of a crude MDA mixture comprising MMDA and PMDA by reaction of aniline with formaldehyde, with the reaction conditions being selected so that the resulting crude MDA can be converted into the gas phase,
(2) conversion of the crude MDA mixture from step (1) into the gas phase and
(3) phosgenation of crude MDA in the gas phase to give MMDI and PMDI.

2. The process according to claim 1, wherein the reaction in step (1) is carried out at a ratio of aniline to formaldehyde of from 5.5 to 7.

3. The process according to claim 1 or 2, wherein the crude MDA mixture formed in step (1) has a proportion of
from 88 to 99.9 percent by weight of MMDA and
from 0.1 to 12 percent by weight of PMDA.

4. The process according to any of claims 1 to 3, wherein the crude MDA mixture formed in step (1) has a proportion of
from 95 to 99 percent by weight of MMDA and
from 1 to 5 percent by weight of PMDA.

5. A mixture comprising MMDA and PMDA in which the content of PPMDA is so low that the mixture can be converted into the gas phase at temperatures of from 200 to 600° C. and pressures of from 2 bar to 20 bar.

6. The mixture according to claim 5 which has a content of
from 88 to 99.9 percent by weight of monomeric methylenedi(phenylamines) and
from 0.1 to 12 percent by weight of polymethylene-polyphenylene polyamines.

7. A gaseous mixture comprising
(a) a mixture according to claim 5 or 6 and
(b) an inert medium.

8. The use of a mixture according to any of claims 5 to 7 for preparing isocyanates by means of gas-phase phosgenation.
